(19) United States
(12) Patent Application Publication
(10) Pub. No.: US 2006/0030549 A1
(43) Pub. Date: Feb. 9, 2006

(54) Paricalcitol(zemplar) capsule controls secondary hyperparathyroidism (SHPT) in CKD stage 5 patients

(76) Inventors: Ping Qiu, Gurnee, IL (US); Joel Z. Melnick, Wilmette, IL (US); Laura A. Williams, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/002,159

(22) Filed: Dec. 2, 2004

Related U.S. Application Data
(60) Provisional application No. 60/527,582, filed on Dec. 5, 2003.

(51) Int. Cl. A61K 31/59

(52) U.S. Cl. 514/167

(57) ABSTRACT

Oral paricalcitol for sustained reduction of parathyroid hormone in dialysis patients.

### CROSS REFERENCE TO RELATED APPLICATIONS

The present application claims priority to U.S. Provisional Application No. 60/527,582, filed on Dec. 5, 2003, hereby incorporated in its entirety by reference

### DESCRIPTION OF THE INVENTION

Paricalcitol capsule is an oral formulation of paricalcitol injection, a vitamin D analog effective in the prevention and treatment of SHPT associated with chronic renal failure. Three doubleblind, placebo-controlled, multicenter studies were conducted to evaluate the safety and efficacy of paricalcitol capsule in CKD stage 5 subjects on hemodialysis (HD) or peritoneal dialysis (PD). After a 4-8 week washout period, 225 subjects (HD: 150, PD: 75) with iPTH≧300 pg/mL, serum Ca 8.0-10.5 mg/dL and Ca×P04<65 were randomized (1:1) and treated with paricalcitol capsule or placebo 3× weekly for 12 weeks. Initial doses were based on iPTH levels at baseline (initial mcg dose=iPTH/60). Subsequent dose adjustments of 2 mcg were based on the weekly Ca, Ca×P04, and iPTH results; dose increases occurred every 2 weeks and decreases once per week.

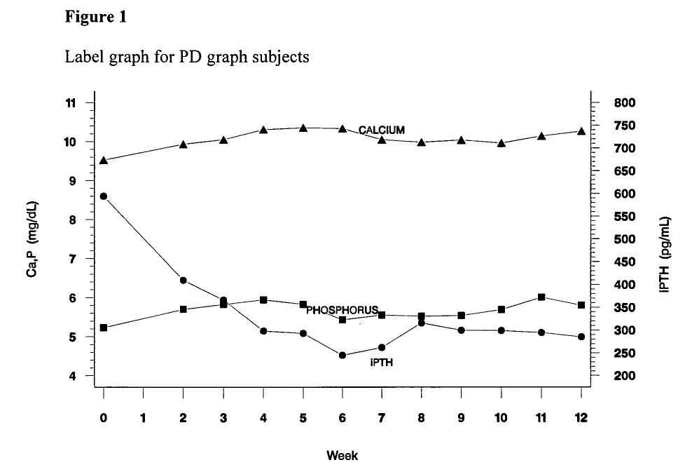

Overall, 95/105 (90%) of paricalcitol-treated subjects had 2 consecutive 30 decreases in iPTH compared to 7/108 (6%) of placebo subjects (p<0.001) (FIG. 1). Efficacy was not influenced by demographics, disease severity, baseline P04 or types of phosphate binder usage. Paricalcitoltreated subjects had a 30% mean iPTH reduction by Week 3 and the reduction was sustained throughout the treatment, while mean Ca remained under 10.0 mg/dL. Mean serum P04 increased initially to a maximum value of 5.7 mg/dL and then decreased. The AE profile was comparable between the groups.

### CONCLUSION

In conclusion, paricalcitol capsule provides sustained reduction of PTH in HD and PD subjects with adverse event profile comparable to placebo. It may be particularly beneficial to PD patients in regular IV administration of paricalcitol is practical.

What is claimed is:

1. Any member of a family of oral formulations comprising:
a therapeutically effective amount of paricalcitol dissolved in an amount of a non-polar solvent, wherein each family member comprises a ratio of non-polar solvent to paricalcitol and said ratio does not vary by more than a factor of about 3.5 to a ratio of non-polar solvent to paricalcitol in a selected referenced oral formulation that is a member of the family and each family member is bioequivalent to the selected referenced formulation and to one another.
